(19) United States
(12) Patent Application Publication
(10) Pub. No.: US 2022/0070993 A1
(43) Pub. Date: Mar. 3, 2022

(54) METHOD TO PRODUCE LIGHT HYDROCARBONS BY COx HYDROGENATION IN A DIELECTRIC BARRIER DISCHARGE PLASMA REACTOR SYSTEM

(71) Applicant: Sogang University Research Foundation, Seoul (KR)

(72) Inventors: Kyoung-Su HA, Hanam-si (KR); Jong Hyun JEON, Seoul (KR); Jaekwon JEOUNG, Seoul (KR)

(73) Assignee: Sogang University Research Foundation, Seoul (KR)

(21) Appl. No.: 17/288,114

(22) PCT Filed: Oct. 25, 2019
(86) PCT No.: PCT/KR2019/014141

§ 371 Date: Nov. 3, 2021

(30) Foreign Application Priority Data
Oct. 25, 2018 (KR) 10-2018-0128519

(51) Int. Cl. H05H 1/24; B01J 37/02; B01J 37/16; B01J 19/08; B01J 8/00; C07C 1/02; B01J 23/89; B01J 35/00

(52) U.S. Cl. CPC H05H 1/2406; B01J 37/0201; B01J 37/16; B01J 19/088; B01J 8/008; B01J 21/08; B01J 23/8913; B01J 35/006; B01J 2219/0894; B01J 2219/00051; C07C 1/02

(57) ABSTRACT

The present invention relates to a dielectric barrier discharge (DBD) plasma reactor comprising a catalyst bed for CO_{X} hydrogenation in a discharge region; and a method to produce light hydrocarbons from a CO_{X}-containing gas mixture in the DBD plasma reactor. In the DBD plasma reactor for a CO_{X} hydrogenation reaction, the catalyst for CO_{X} hydrogenation comprises a catalytically active component on a mesoporous support that is a dielectric. When the DBD plasma reactor for a CO_{X} hydrogenation reaction according to the present invention is used, it is possible to convert by-product gases or waste gases into higher-value-added chemical products without additional heat supply from the outside.

### TECHNICAL FIELD

The present invention relates to a dielectric barrier discharge (DBD) plasma reactor comprising a catalyst bed for CO_{X} hydrogenation in a discharge region; and a method to produce light hydrocarbons from a CO_{X}-containing gas mixture in the DBD plasma reactor.

### BACKGROUND ART

Methane is a main component of natural gas resources, and thus a technology to convert methane into more valuable hydrocarbons and fuels is of the utmost importance. There have been extensive research efforts to utilize a large number of natural gas reservoirs by using efficient catalysts and various conversion technologies. For economic reasons, large gas fields have been developed and used for the gas-to-liquid process, methanol-to-olefin process, methanol synthesis, dimethyl ether synthesis, and the like.

These technologies involve energy-intensive steps such as gasification and reforming reactions, and are usually carried out at significantly high temperatures. Severe reaction conditions may limit the choice of reactor materials and reaction catalysts. Because of this situation, it is difficult to achieve optimal reaction conditions and to implement or operate the reactors with the best design.

In general, the chemical conversion technology to obtain hydrocarbons through hydrogenation of CO_{X} gas is a kind of indirect conversion technology using syngas as a basic reactant. Typical technologies are a methanol synthesis technology carried out at 50 atm or more and a reaction temperature of 250° C. or more on a catalyst composed of constituents such as copper, zinc, and alumina, the Fischer-Tropsch synthesis technology carried out under conditions of 10 atm to 30 atm and 220° C. to 350° C. on cobalt-based and iron-based catalysts, a synthetic natural gas production technology carried out at 300° C. or more on a nickel-based catalyst, and the like. Most of the CO_{X} hydrogenation reactions are required to be performed at high temperatures and high pressures, and a great amount of heat is required to be supplied in order to overcome the activation energy of the conversion reaction.

Meanwhile, by-product gases obtained from the steel industry or the chemical industry are mixtures of carbon monoxide, carbon dioxide, hydrogen, methane, and the like, and the potential for utilization thereof is significantly high, but the by-product gases are called BFG (blast furnace gas), LDG (Linz-Donawitz converter gas), COG (coke oven gas), FOG (finex off gas), and the like, are composed of different components from source to source, contain heavy metals, dust, and catalyst poisons, and thus are mainly recovered as heat since there are a number of restrictions on the method for converting the by-product gases into high-value-added compounds.

Meanwhile, ozone generation was first introduced in the mid-19th century by the non-thermal dielectric barrier discharge (DBD) plasma method. Recently, it has been reported that high-value-added products are produced using a conversion method by way of plasma.

### SUMMARY OF INVENTION

### Technical Problem

The present invention is to propose a method for converting the above-described by-product gases or waste gases into higher-value-added chemical products without additional heat supply from the outside. The compounds obtained by the present invention are mainly composed of methane of a synthetic natural gas, ethane of a reactant in ethane cracking, and propane and butane of LPG components.

### Solution to Problem

A first aspect of the present invention provides a dielectric barrier discharge (DBD) plasma reactor for a CO_{X} hydrogenation reaction, comprising a catalyst bed for CO_{X} hydrogenation in a discharge region, wherein the catalyst for CO_{X} hydrogenation comprises a catalytically active component on a mesoporous support that is a dielectric.

A second aspect of the present invention provides a method to produce light hydrocarbons from a CO_{X}-containing gas mixture in a dielectric barrier discharge (DBD) plasma reactor comprising a catalyst bed for CO_{X} hydrogenation in a discharge region, which comprises: a first step of reducing a metal-based catalytically active component at 300° C. to 500° C. in a reducing atmosphere to preliminarily activate a catalyst for CO_{X} hydrogenation; and a second step of forming light hydrocarbon(s) in gas-phase through plasma conversion of CO_{X} without heat supply from the outside.

A third aspect of the present invention provides a method to produce a high-value-added chemical product comprising a step of converting a by-product gas or a waste gas into a high-value-added chemical product in the dielectric barrier discharge (DBD) plasma reactor for CO_{X} hydrogenation reaction according to the first aspect without additional heat supply from the outside.

A fourth aspect of the present invention provides a method to remove CO₂ from a CO_{X}-containing gas mixture without CO removal, comprising a step of forming dielectric barrier discharge plasma on a catalyst bed in the dielectric barrier discharge (DBD) plasma reactor for a CO_{X} hydrogenation reaction according to the first aspect without heat supply from the outside, wherein the catalyst bed comprises a catalyst for CO_{X} hydrogenation which is not activated preliminarily by reducing a catalytically active component based on transition-metal, or a mesoporous support only that is a dielectric and does not comprise a metal-based active component.

### Advantageous Effects of Invention

When the DBD plasma reactor for the CO_{X} hydrogenation reaction according to the present invention is used, it is possible to convert by-product gases or waste gases into higher-value-added chemical products without additional heat supply from the outside.

### BRIEF DESCRIPTION OF DRAWINGS

FIG. 1 schematically illustrates an apparatus equipped with a DBD plasma reactor according to an embodiment of the present invention.

FIG. 2 is analysis results of the small-angle XRD and wide-angle XRD of produced support SBA-15 and catalyst 0.1Pt-20Co@SBA-15.

FIG. 3 is a graph illustrating the conversion performance and selectivity during plasma conversion at room temperature in a reactor packed with produced support SBA-15 and catalyst 0.1Pt-20Co@SBA-15.

FIG. 4 is a schematic diagram of a DBD plasma reactor which comprises an external furnace and a heat insulating material to maintain an adiabatic condition.

FIG. 5 illustrates the time courses of CO and CO₂ conversions during a plasmatic reaction at room temperature using a reactor without packing of a catalyst or an SBA-15 support according to Comparative Example 1.

FIG. 6 is a schematic diagram of a thermochemical reaction apparatus using a catalyst 0.1Pt-20Co@SBA-15.

FIG. 7 illustrates the GC analysis results during a thermochemical FTS (high temperature) reaction in a reactor packed with 0.1Pt-20Co@SBA-15.

FIG. 8 illustrates the GC analysis result during a thermochemical FTS (low temperature) reaction in a reactor packed with 0.1Pt-20Co@SBA-15.

### DESCRIPTION OF EMBODIMENTS

Hereinafter, the present invention will be described in detail.

In the present invention, dielectric barrier discharge plasma is used as an energy source for converting reactive molecules into light hydrocarbons.

Dielectric barrier discharge (DBD) refers to the electrical discharge between two electrodes separated by an insulating dielectric barrier. DBD is also called silent or inaudible discharge and is also known as ozone production discharge or partial discharge. For example, an alumina tube may be used as a dielectric barrier.

Dielectric barrier discharge is widely used in industry since DBD can be generated at atmospheric pressure and room temperature, operates in significantly large non-equilibrium conditions at atmospheric pressure, can perform high-output discharge, and does not require a complex pulse power supply.

Low-temperature plasma having a generated electron temperature relatively higher than the temperature of gas may be formed through dielectric barrier discharge (DBD).

The dielectric barrier discharge plasma reactor may be equipped with: (a) a tubular container that is made of a dielectric material and can usually accommodate a catalyst; (b) a ground electrode disposed on the outer wall of the tubular container; (c) a high-voltage electrode to which a voltage higher than that of the ground electrode is applied and which is inserted into the catalyst accommodated in the tubular container so as to be spatially spaced parallel to the tubular container made of a dielectric material; (d) a fixing unit for locating and fixing the catalyst which is contained in the tubular container and used in the reaction at a predetermined region; and (e) a power supply unit for providing a regulated voltage to the high-voltage electrode.

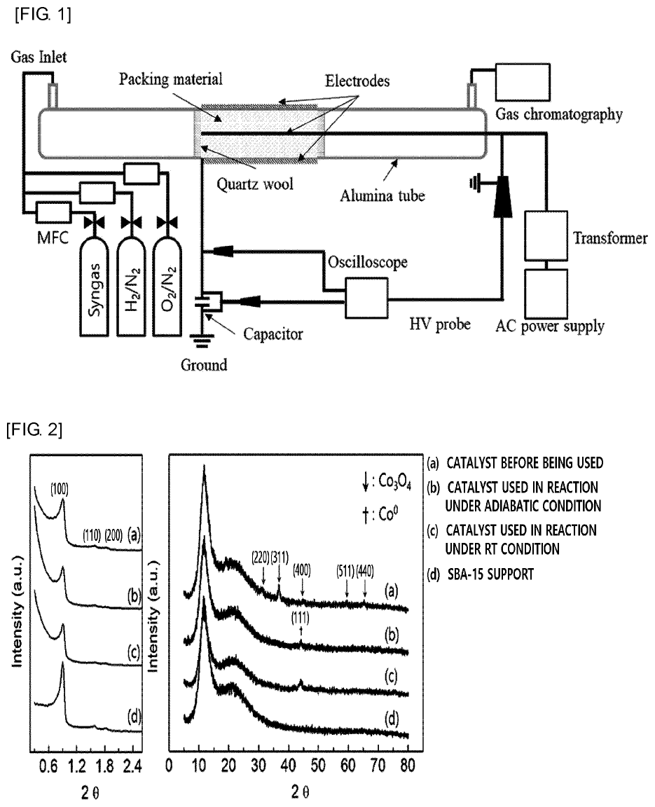

FIG. 1 1 schematically illustrates an apparatus equipped with a DBD plasma reactor according to an embodiment of the present invention.

In the dielectric barrier discharge (DBD) plasma reactor comprising a catalyst bed for CO_{X} hydrogenation in the discharge region according to the present invention, the catalyst for CO_{X} hydrogenation comprises a catalytically active component on a mesoporous support that is a dielectric.

In the present invention, the mesoporous support is preferably an ordered mesoporous support. This is because the metal catalyst material can be uniformly supported on the support. Non-limiting examples of the mesoporous support that is a dielectric include ordered mesoporous silica (OMS).

By application of the DBD plasma reactor comprising a catalyst bed for CO_{X} hydrogenation comprising a catalytically active component on a mesoporous support that is a dielectric of the present invention, the conversion rate of reaction may be further increased by using adiabatic conditions even though heat is not supplied from the outside, high CO conversion rate is achieved unlike the results under RT conditions, and CO₂ conversion rate similar to the results under RT conditions may be achieved.

For example, the catalyst for CO_{X} hydrogenation may be obtained by impregnating a catalytically active component into the pores of a mesoporous support by an incipient wetness impregnation method using an aqueous solution of precursor(s) of the catalytically active component.

Microelectrodes are induced between dielectric particles (namely, the catalyst particles for CO_{X} hydrogenation comprising a catalytically active component on a mesoporous support that is a dielectric according to the present invention) charged by an external electric field. Streamers and microdischarges are generated due to high voltages and dielectric barriers. These streamers and microdischarges reach the upper surface of one particle, and this particle is positively charged due to polarization. At the same time, the lower surface of the particle is negatively charged. The upper surface and the lower surface of the particle become an anode-like surface and a cathode-like surface, respectively. This phenomenon starts from the particles near the outer cathode, continuously occurs from particles to particles, and proceeds toward the opposite electrode. When the streamers surround the dielectric particles, the intensity of the local electric field is strengthened by photo-ionization, and thus electrons are sprinkled like seeds from the lower surface of the particle. The electrons sprinkled like seeds cause another avalanche and start new streamers. At this moment, the reactive molecules collide with the accelerated electrons, and positively charged ions are thus generated. The cations generated in the strengthened electric field are accelerated to move to the cathode-like surface and collide with the surface. This collision generates secondary electrons to sustain the streamers. Hence, the induced local electric field and the charged surface of the induced particles may be regarded as microelectric fields and microelectrodes, respectively.

Hence, the gap distance between dielectric particles in the catalyst bed for CO_{X} hydrogenation comprised in the discharge region may be adjusted so as to have a low breakdown voltage in the DBD plasma reactor. The average gap distance of the catalyst particles comprising dielectric mesoporous supports may be 1 μm to 20 μm. Through experiments, the gap distance between dielectric particles may be determined according to the sizes of the dielectric particles, and the average particle size of catalyst particles may be in a microscale range of 10 μm to 200 μm.

In the complex system of the plasma and catalyst, they may interact with each other to increase the efficiency of reaction and improve the selectivity of product. Non-limiting examples of usable catalysts include noble metals, transition metals, and typical metals as active materials. In particular, active materials may include Pt, Ru, Ni, Co, V, Fe, Cu, Ti, Nb, Mo, W, Ta, Pd, Cu, or Zn, and active materials or carriers may include transition metal oxides such as ZrO₂, CoO, Co₃O₄, MnO, NiO, CuO, ZnO, TiO₂, V₂O₅, Ta₂O₅, ZnO, Cr₂O₃, FeO, Fe₂O₃, and Fe₃O₄ and oxides of typical elements such as MgO, CaO, BaO, Al₂O₃, Ga₂O₃, SnO, and SnO₂. The usable support that is a dielectric may include silica (SiO₂), zeolite, mesoporous materials, activated carbon, layered double hydroxides (LDH), and the like.

The catalyst may be in the form of a sphere, a pellet, a monolith, a honeycomb, a fiber, a porous solid foam, and a powder. The catalyst in the above form may be packed inside the plasma reactor to form a packed-bed reactor. The catalyst may be coated on the inner wall of the reactor performing a plasma-catalyst reaction to form a catalyst bed.

For example, the catalyst bed for CO_{X} hydrogenation may be packed with catalyst particles in which a transition metal (Co, Fe, Ni, Ru, or the like) as a main catalytically active component is supported on a porous inorganic support.

The plasma-catalyst reactor may be operated at a relatively low temperature (for example, on the order of hundreds of K to 1000 K) by using a catalyst together therewith. In the present invention, the CO_{X} conversion reaction by plasma may be performed at room temperature or 200° C. or less.

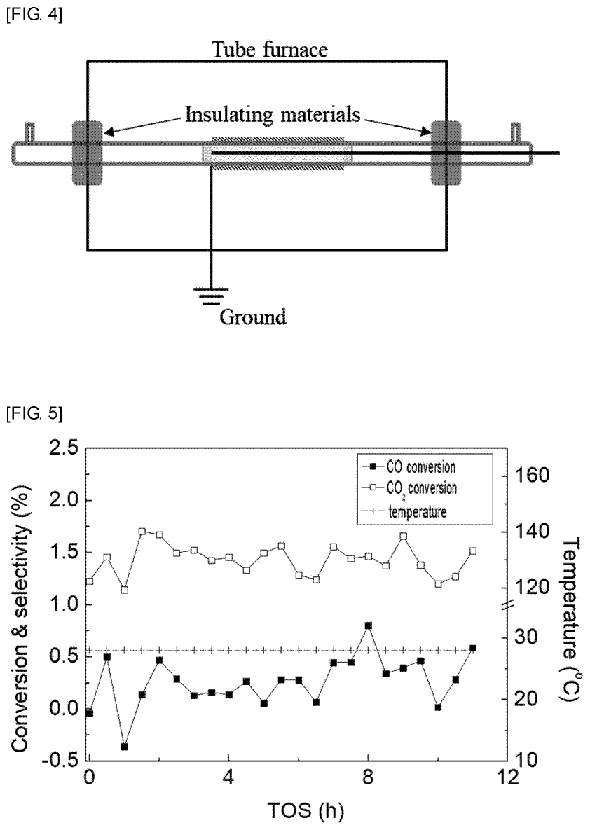

The DBD plasma reactor for CO_{X} hydrogenation reaction of the present invention can perform synthesis at normal pressure, and thus the reactor configuration is relatively simple, and the reactor may be designed to have various structures. For example, the reactor may be designed so that the CO_{X} hydrogenation reaction is performed under adiabatic conditions (FIG. 4). For example, the temperature may be raised to about 150° C. by insulating the heat generated during the reaction.

The DBD plasma reactor for the CO_{X} hydrogenation reaction of the present invention may be designed so that the metal-based catalytically active component in the catalyst bed for CO_{X} hydrogenation can be reduced at a high temperature.

The present invention relates to a method to produce light hydrocarbons by activating a CO_{X}-containing mixture (non-limiting examples thereof include a by-product gas such as COG) of a reactant on a catalyst using dielectric barrier discharge (DBD) plasma that is a kind of low-temperature plasma.

The method to produce light hydrocarbons from a CO_{X}-containing gas mixture in a dielectric barrier discharge (DBD) plasma reactor comprising a catalyst bed for CO_{X} hydrogenation in a discharge region according to the present invention includes:

a first step of reducing a metal-based catalytically active component at 300° C. to 500° C. in a reducing atmosphere such as a hydrogen atmosphere to preliminarily activate a catalyst for CO_{X} hydrogenation; and

a second step of forming light hydrocarbon(s) in gas-phase through plasma conversion of CO_{X} without heat supply from the outside.

The present invention is, for example, the use of dielectric barrier discharge plasma as an energy source for converting reactive molecules into light hydrocarbons by activating the reduced transition metal catalyst that is a catalyst for hydrogenation and the reactive molecules.

At this time, the DBD plasma reactor used in the method to produce light hydrocarbons of the present invention may be the DBD plasma reactor of the present invention described above as the catalyst for CO_{X} hydrogenation comprises a catalytically active component on a mesoporous support that is a dielectric.

If the metal-based catalytically active component is not preliminarily activated through high-temperature reduction in a reducing atmosphere in the first step, the CO conversion rate is zero, and CO₂ is mainly converted in the conversion reaction by the catalyst-plasma in the second step. In other words, it can be seen that a reduced or activated metal component on the dielectric particles is required for the CO conversion rate to be >0 in the CO_{X} hydrogenation reaction by DBD plasma.

If the first step is carried out in a DBD plasma reactor, the reactor may be purged with a reaction gas at room temperature before the second step.

In the second step, CO_{X} in the mixture for reaction is hydrogenated on the catalyst, and the reaction proceeds even at a low temperature close to room temperature if the dielectric barrier discharge is used as a method for supplying the activation energy, and a higher reaction conversion rate may be obtained by using adiabatic conditions even though heat is not supplied from the outside.

Therefore, the second step is achieved by performing a plasma converting reaction of CO_{X} at room temperature or 200° C. or less. The reaction of the second step may be performed as a room-temperature (RT) reaction or an adiabatic reaction performed under adiabatic conditions. There are a synthetic natural gas (SNG) production process and the Fischer-Tropsch synthesis (FTS) process as methods for obtaining hydrocarbons by hydrogenation of CO_{X} gas. The former mainly synthesizes a methane component and involves tremendous heat generation. The latter mainly produces gasoline, naphtha, and diesel and also involves tremendous heat generation. In addition, a significant amount of heat is required to be provided to the reactor to activate the reaction. In the present invention, the conversion reaction can take place even at a significantly low temperature when compared with the above hydrocarbon production technology, the reaction can be performed at 200° C. or less even under adiabatic conditions, and it is not required to supply heat from the outside. When the reaction of the second step is performed as an adiabatic reaction, the CO conversion rate can be increased through insulation, but the CO conversion and the CO₂ conversion are inversely proportional to each other.

Synthesis can be performed at normal pressure, and thus the reactor configuration is relatively simple, and the reactor may be designed to have various structures.

In the second step, the space velocity during the reaction may be 2000 mL/g/h to 12000 mL/g/h.

The method to produce light hydrocarbons according to the present invention may be used to synthesize light hydrocarbons from a by-product gas, a syngas mixture, and the like that are gas mixtures containing CO_{X}.

In the present invention, DBD plasma is used, and thus a CO_{X}-containing gas mixture which contains heavy metals, dust, and/or catalyst poisons may also be used as a reaction gas. For example, the CO_{X}-containing gas mixture may be a by-product gas obtained from the steel industry or the chemical industry. Non-limiting examples of the by-product gas include BFG (blast furnace gas), LDG (Linz-Donawitz converter gas), COG (coke oven gas), and FOG (finex off gas). For example, the CO_{X}-containing gas mixture may be an industrial by-product gas containing carbon monoxide, carbon dioxide, hydrogen, and methane.

The product obtained from the reaction in the second step is light hydrocarbons in gas-phase, mainly saturated hydrocarbons such as methane, ethane, propane, butane, and pentane, and olefin hydrocarbons that are inevitably obtained in the reaction pathway are also produced in small amounts.

As the composition of the product gas obtained in the present invention, not only methane but also ethane, propane, butane, pentane, and the like that exist in the gaseous phase at room temperature and normal pressure are obtained, but liquid or solid products are hardly synthesized.

In the present invention, light hydrocarbons (C₂ to C₄ paraffin and olefin compounds) may be continuously produced through the second step.

The C₂₊ hydrocarbons may be used as a raw material to be converted into high-value-added chemical products and higher-heating-value fuels.

Therefore, in the present invention, it is possible to convert by-product gases or waste gases into high-value-added chemical products in a dielectric barrier discharge (DBD) plasma reactor for a CO_{X} hydrogenation reaction without additional heat supply from the outside.

Moreover, CO₂ can be removed from a CO_{X}-containing gas mixture without CO removal when the dielectric barrier discharge (DBD) plasma reactor for CO_{X} hydrogenation reaction according to the present invention is used, but the catalytically active component that is a transition metal is not reduced to preliminarily activate the catalyst for CO_{X} hydrogenation, or only a mesoporous support that is a dielectric and does not support a metal-based active component is used, and dielectric barrier discharge plasma is formed on the catalyst bed without heat supply from the outside.

### EMBODIMENTS

Hereinafter, the present invention will be described in more detail with reference to Examples. However, these Examples are for illustrative purposes only, and the scope of the present invention is not limited to these Examples.

### Preparation Example 1: Fabrication of DBD Plasma Reactor

The DBD plasma reaction apparatus used in the present invention will be schematically described below. The DBD plasma reaction apparatus was configured as illustrated in FIG. 1. As the dielectric barrier reaction tube, an alumina tube having an outer diameter of 10 mm and an inner diameter of 6 mm was used. Stainless steel was used as the high-voltage electrode and located in the center of the reaction tube. The catalyst or SBA-15 support was packed in a length of 5 cm at the portion where the high-voltage electrode was located in the dielectric barrier reaction tube. A sinusoidal AC power supply (0 V to 220 V, 60 Hz to 1000 Hz) was connected to a transformer (0 kV to 20 kV, 1000 Hz), and a high voltage was continuously applied to the plasma bed by the electrical system. The voltage applied to the plasma bed was 15 kV, the frequency was fixed at 1000 Hz, and a capacitor having a capacitance of 1 μF was connected in series between the plasma bed and the ground connection. A high-voltage probe (1000:1, P6015A, Tektronix) was installed on the high-voltage electrode, and a current probe (TCP202, Tektronix) was installed on the ground electrode. Probes (10:1, P6100, Tektronix) were connected to both ends of the battery. All probes were connected to a digital oscilloscope (TDS 3013C, Tektronix) to observe the respective voltage and current data in real time.

Each of the packing materials was packed in a mass of 0.2 g to 0.3 g in the discharge region, and the space velocity during the reaction was maintained at 4000 mL/g/h. The reaction product was analyzed using online gas chromatography (6500GC Young Lin Instrument Co., Korea). For online GC, Porapak-N and Molecular Sieve 13X column connected to a thermal conductivity detector (TCD) and a Gas-pro column connected to a flame ionization detector (FID) were used. H₂, Ar, CH₄, CO, and CO₂ in the product were detected by TCD, and hydrocarbons such as CH₄, C₂H₆, C₂H₄, C₃H₈, C₃H₆, n-C₄H₁₀, and 1-C₄H₈ were detected by FID. The conversion rates were calculated through the changed amounts of CO and CO₂ with respect to the input amounts of CO and CO₂. The selectivities were calculated through the numbers of moles of the substances converted from CO and CO₂ with respect to the total numbers of carbon atoms converted from CO and CO₂.

### Preparation Example 2: Synthesis Method and Characteristic Analysis Result of 0.1Pt-20Co/SBA-15

In order to provide ordered mesoporous silica (OMS) as a support for catalyst synthesis, SBA-15 was produced by slightly modifying a method known in the art. Specifically, 12 g of P123 (EO₂₀PO₇₀EO₂₀, MW=5,800 g/mol) was dissolved in 450 g of a 1 M HCl solution at 36° C. After 24 hours, it was confirmed that P123 was uniformly dissolved, and then 25.76 g of TEOS (tetraethyl orthosilicate) was added thereto dropwise, and mixed for 24 hours. The mixture was placed in a PP bottle and underwent hydrothermal synthesis in an oven at 100° C. for 24 hours. The product was washed with water and ethanol three times and then dried in an oven at 110° C. for 12 hours, and the dried powder material was calcined at 500° C. for 5 hours to obtain the title compound.

The produced SBA-15 was dried in an oven at 110° C. for 12 hours or more to remove residual moisture. In order to put an aqueous solution of cobalt and platinum precursors into the pores of SBA-15, 1.26 g of cobalt nitrate hexahydrate (Co(NO₃)₂.6H₂O) and 0.002 g of tetraammineplatinum(II) nitrate (Pt(NH₃)₄(NO₃)₂) were mixed with and completely dissolved in 3 mL of distilled water. Thereafter, the aqueous solution of cobalt and platinum precursors was impregnated into the pores of the dried SBA-15 powder little by little by way of an incipient wetness impregnation method. The SBA-15 powder in which the aqueous solution of precursor(s) was well impregnated into the pores was sufficiently dried in an oven at 110° C. for 10 to 24 hours to remove water from the aqueous solution of precursor(s). The dried powder was heated to 400° C. at a rate of temperature rise of 2° C./min, and then maintained at the same temperature for 5 hours for calcination. By performing the above process, a 0.1Pt-20Co@SBA-15 catalyst was obtained.

The small-angle XRD and wide-angle XRD analysis results of the produced SBA-15 support and 0.1Pt-20Co@SBA-15 catalyst are illustrated in FIG. 2. As a small-angle XRD analysis result of the support and catalyst, it was confirmed that mesopores were well ordered through reflection planes (100), (110), and (200). It was confirmed that Co₃O₄ crystals were successfully produced through wide-angle XRD analysis of the produced catalyst. The average size of Co₃O₄ particles obtained from the 36.7° (311) plane result by way of the Scherrer equation was 13.4 nm.

The particle sizes of Co₃O₄ and Co⁰ calculated based on the analysis results of the produced catalyst and the catalyst after a reaction are presented in Table 1. The d-spacing between planes (100) obtained as a result of small-angle XRD analysis was calculated and is also presented in Table 1.

TABLE 1

| | Wide-angle XRD | | Small-angle |
|---|---|---|---|
| | Co₃O₄ crystal | Co⁰ crystal | XRD d-spacing |
| Name | size (nm) | size (nm) | (nm) |
| Catalyst before being used | 13.4 | — | 9.8 |
| Catalyst used in reaction | — | 22.3 | 9.8 |
| under adiabatic condition | | | |
| Catalyst used in reaction | — | 8.8 | 9.8 |
| under RT condition | | | |
| SBA-15 support | — | — | 9.8 |

### Example 1: Plasma Conversion at Room Temperature in Reactor Packed with 0.1Pt-20Co@SBA-15 Catalyst

The 0.1Pt-20Co@SBA-15 catalyst was loaded into the DBD plasma reactor configured in Preparation Example 1, and a reaction experiment was performed under a room-temperature (RT) condition. The length of the catalyst bed packed was 5 cm, and the catalyst was evenly packed in this region and fixed in the reaction space with quartz wool. Before the reaction, the catalyst was reduced in a hydrogen atmosphere using an external furnace to perform a preliminary activation process. As the reducing gas, prepared gas of 5% H₂/N₂ was used. The space velocity was maintained at 4000 mL/g/h. The temperature during the reduction was raised at a rate of 2° C./min, and then maintained constant at 400° C. for 6 hours. After the reduction was completed, the reactor was cooled to room temperature and then purged with the reaction gas.

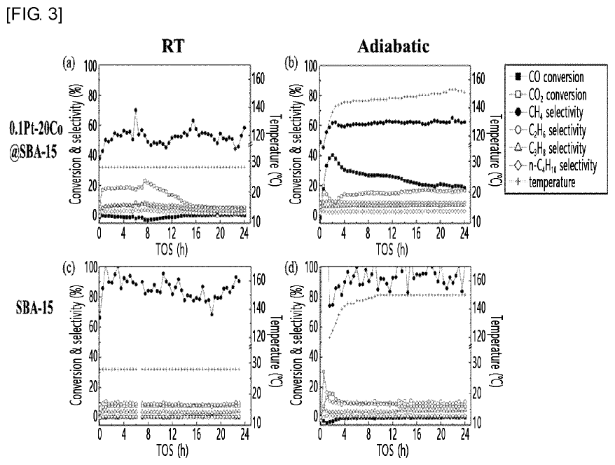

The reaction gas (feed) was a prepared gas having a molar ratio of 58% hydrogen, 28.1% carbon monoxide, 4.8% argon, and 9.1% carbon dioxide. The reaction was performed by maintaining the gas hourly space velocity (GHSV) at 4000 mL/g/h. This gas hourly space velocity corresponds to a flow rate of 13.33 mL/min. The reaction was sustained by continuously applying a sinusoidal high voltage of 15 kV at 1 kHz for the plasma reaction. During the reaction, the conversion performance and selectivity were observed using online gas chromatography. The results are illustrated in FIG. 3-(a).

### Example 2: Plasma Conversion Under Adiabatic Condition in Reactor Packed with 0.1Pt-20Co@SBA-15 Catalyst

As illustrated in FIG. 4, an external furnace and a heat insulating material were used to maintain an adiabatic condition. The reaction conditions other than this were the same as those in Example 1. It was possible to raise the temperature to about 150° C. by insulating the heat generated during the reaction even without a separate external heat source. Unlike the results of the experiment under a RT condition, a high CO conversion rate was observed, and the CO₂ conversion was also maintained at about 15%. It was observed that the CO conversion rate and the CO₂ conversion rate were inversely proportional to each other. The detailed reaction results are illustrated in FIG. 3-(b).

### Comparative Example 1: Plasma Conversion at Room Temperature without Catalyst Packing

A reaction performance test was performed in the same reaction gas composition and under the same flow condition as in Example 1 without packing any material inside the reactor. In the absence of catalyst or dielectric, CO and CO₂ conversion hardly took place, and the time courses of CO and CO₂ conversions are illustrated in FIG. 5.

### Comparative Example 2: Plasma Conversion at Room Temperature in Reactor Packed with SBA-15 Dielectric

The SBA-15 dielectric was packed in the reactor, and a reaction performance test was performed under the same conditions as in Example 1. Preliminary activation through reduction was not performed. The CO conversion rate was 0, and the CO₂ conversion was observed to be about 7%. The detailed GC analysis results are illustrated in FIG. 3-(c).

### Comparative Example 3: Plasma Conversion Under Adiabatic Condition in Reactor Packed with SBA-15 Dielectric

The SBA-15 dielectric was packed in the reactor, and a reaction performance test was performed under the same conditions as in Example 2. Preliminary activation through reduction was not performed. The GC analysis results are illustrated in FIG. 3-(d).

### Comparative Example 4: Thermochemical FTS (High Temperature) in Reactor Packed with 0.1Pt-20Co@SBA-15

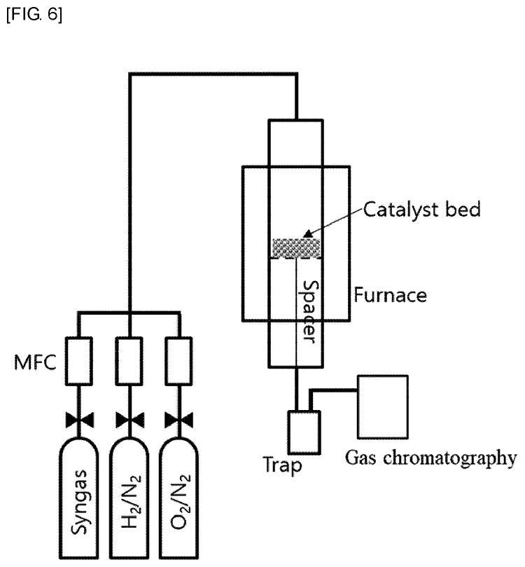

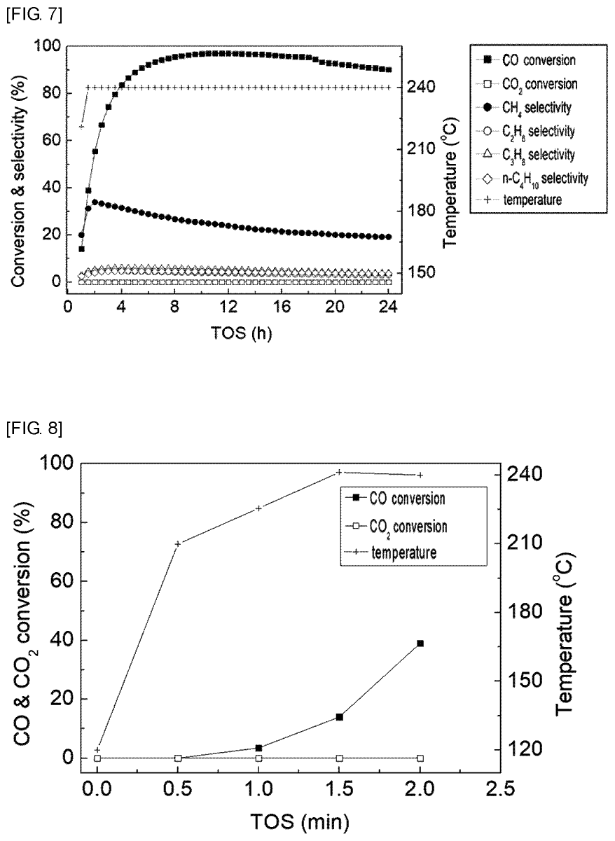

In order to examine the difference in reactivity of 0.1Pt-20Co@SBA-15 between the plasma reaction and the thermochemical reaction, a thermochemical reaction experiment was performed in the reaction apparatus as illustrated in FIG. 6. As a platinum precursor for catalyst production, diaminedinitritoplatinum(II) (Pt(NH₃)₂(NO₂)₂) was used. As the cobalt precursor, cobalt nitrate hexahydrate (Co(NO₃)₂.6H₂O) was used. The two precursors were dissolved in ethanol and supported on SBA-15 by an incipient wetness impregnation method. Although the solvent used for the platinum precursor is slightly different from that in Preparation Example 1, the properties and compositions of the finally obtained catalysts are quite similar to each other, and therefore the catalyst is sufficient to be used as in the Comparative Examples. A preliminary activation process was performed as in Example 1, and then the pressure and the temperature were sequentially raised to 20 bar and 240° C. to perform the reactivity test. The GC analysis results during the reaction are illustrated in FIG. 7.

### Comparative Example 5: Thermochemical FTS (Low Temperature) in Reactor Packed with 0.1Pt-20Co@SBA-15

In order to compare the activities when thermochemical FTS was performed at low temperatures, the same catalyst as in Comparative Example 4 was produced and used. The reactor illustrated in FIG. 6 was utilized under the same reaction conditions as in Comparative Example 4, and the GC analysis results at this time are illustrated in FIG. 8. It has been confirmed that the CO conversion rate is 0 in a low temperature range of less than 210° C.

### Example 3: Performance Improvement by Catalyst Material in Plasma Reaction at Room Temperature (Comparison of Example 1 with Comparative Example 2)

The activity for CO₂ conversion in the case of using a catalyst at RT (Example 1) is higher than that in the case of using a simple SBA-15 dielectric (Comparative Example 2) by about 10%. In both cases, the activity for CO conversion is not observed.

### Example 4: Performance Improvement by Catalyst Material in Plasma Reaction Under Adiabatic Condition (Comparison of Example 2 with Comparative Example 3)

When the plasma reaction is performed using a catalyst under an adiabatic condition, CO can be effectively converted. The maximum conversion rate was 40%, the conversion rate was stabilized at about 30% thereafter, and the catalyst was then gradually deactivated. This is greatly different from the CO conversion rate of 0% when only the SBA-15 dielectric is used, and the CO₂ conversion rate is also similar to or higher than the CO₂ conversion when only the SBA-15 dielectric is used.

### Example 5: Comparison of Plasma Conversion Under Adiabatic Condition in Reactor Packed with 0.1Pt-20Co@SBA-15 (Example 2) with Thermochemical Reaction at High Temperature in Reactor Packed with 0.1Pt-20Co@SBA-15 (Comparative Example 4)

In the case of converting CO and CO₂ under an adiabatic condition using plasma, 20% of CO₂ can be converted, although the CO conversion rate is lower than that in the case of thermochemical FTS. It is greatly advantageous that the reaction in Example 2 can be performed at normal pressure without a separate external heat source, whereas the temperature and the pressure are maintained at 240° C. and 20 bar, respectively, during the FTS reaction in Comparative Example 4.

### Example 6: Comparison of Plasma Conversion Under Adiabatic Condition in Reactor Packed with 0.1Pt-20Co@SBA-15 (Example 2) with Thermochemical Reaction at Low Temperature in Reactor Packed with 0.1Pt-20Co@SBA-15 (Comparative Example 5)

In Comparative Example 5, it has been confirmed that CO and CO₂ are hardly converted at temperatures equal to or less than a certain level during the FTS reaction. Hence, the CO and CO₂ conversion rates in Example 2 are considered to be due to a synergistic effect of the interaction between the plasma and the catalyst.

1. A dielectric barrier discharge (DBD) plasma reactor for a CO_{X} hydrogenation reaction, comprising a catalyst bed for CO_{X} hydrogenation in a discharge region,
wherein the catalyst for CO_{X} hydrogenation comprises a catalytically active component on a mesoporous support that is a dielectric.

2. The DBD plasma reactor for a CO_{X} hydrogenation reaction according to claim 1, wherein the mesoporous support is an ordered mesoporous support.

3. The DBD plasma reactor for a CO_{X} hydrogenation reaction according to claim 1, which is designed to operate at normal pressure.

4. The DBD plasma reactor for a CO_{X} hydrogenation reaction according to claim 1, which is designed so that CO_{X} hydrogenation is performed under an adiabatic condition.

5. The DBD plasma reactor for a CO_{X} hydrogenation reaction according to claim 1, which is designed so that a metal-based catalytically active component in the catalyst bed for CO_{X} hydrogenation is reduced at a high temperature.

6. The DBD plasma reactor for a CO_{X} hydrogenation reaction according to claim 1, wherein the catalyst for CO_{X} hydrogenation is obtained by impregnating a catalytically active component into pores of the mesoporous support by an incipient wetness impregnation method using an aqueous solution of precursor(s) of the catalytically active component.

7. The DBD plasma reactor for a CO_{X} hydrogenation reaction according to claim 1, wherein an average particle size of catalyst particles in the catalyst bed for hydrogenation is in a microscale range of 10 μm to 200 μm.

8. The DBD plasma reactor for a CO_{X} hydrogenation reaction according to claim 1, wherein an average gap distance between catalyst particles in the catalyst bed for hydrogenation is 1 μm to 20 μm.

9. A method to produce light hydrocarbons from a CO_{X}-containing gas mixture in a dielectric barrier discharge (DBD) plasma reactor comprising a catalyst bed for CO_{X} hydrogenation in a discharge region, which comprises:
a first step of reducing a metal-based catalytically active component at 300° C. to 500° C. in a reducing atmosphere to preliminarily activate a catalyst for CO_{X} hydrogenation; and
a second step of forming light hydrocarbon(s) in gas-phase through plasma conversion of CO_{X} without heat supply from the outside.

10. The method to produce light hydrocarbons according to claim 9, wherein the dielectric barrier discharge (DBD) plasma reactor is the DBD plasma reactor for a CO_{X} hydrogenation reaction, comprising a catalyst bed for CO_{X} hydrogenation in a discharge region, wherein the catalyst for CO_{X} hydrogenation comprises a catalytically active component on a mesoporous support that is a dielectric.

11. The method to produce light hydrocarbons according to claim 9, wherein the CO_{X}-containing gas mixture contains one or more selected from the group consisting of a heavy metal, dust, and/or a catalyst poison.

12. The method to produce light hydrocarbons according to claim 9, wherein the CO_{X}-containing gas mixture is a by-product gas obtained from a steel industry or a chemical industry.

13. The method to produce light hydrocarbons according to claim 9, wherein the CO_{X}-containing gas mixture is an industrial by-product gas containing carbon monoxide, carbon dioxide, hydrogen, and methane.

14. The method to produce light hydrocarbons according to claim 13, wherein the by-product gas is BFG (blast furnace gas), LDG (Linz-Donawitz converter gas), COG (coke oven gas), or FOG (finex off gas).

15. The method to produce light hydrocarbons according to claim 9, wherein the second step is achieved by performing a plasma converting reaction of CO_{X} at room temperature to 200° C.

16. The method to produce light hydrocarbons according to claim 9, wherein the second step is achieved by performing a plasma converting reaction of CO_{X} at normal pressure.

17. A method to produce a high-value-added chemical product comprising a step of converting a by-product gas or a waste gas into a high-value-added chemical product in the dielectric barrier discharge (DBD) plasma reactor for CO_{X} hydrogenation reaction according to claim 1 without additional heat supply from the outside.

18. A method to remove CO₂ from a CO_{X}-containing gas mixture without CO removal, comprising a step of forming dielectric barrier discharge plasma on a catalyst bed in the dielectric barrier discharge (DBD) plasma reactor for a CO_{X} hydrogenation reaction according to claim 1 without heat supply from the outside,
wherein the catalyst bed comprises a catalyst for CO_{X} hydrogenation which is not activated preliminarily by reducing a catalytically active component based on transition-metal, or a mesoporous support only that is a dielectric and does not support a metal-based active component.

19. The method to produce light hydrocarbons according to claim 9, wherein the DBD plasma reactor is designed so that CO_{X} hydrogenation is performed under an adiabatic condition.

20. The method to produce light hydrocarbons according to claim 10, wherein an average particle size of catalyst particles in the catalyst bed for hydrogenation is in a microscale range of 10 μm to 200 μm and an average gap distance between catalyst particles in the catalyst bed for hydrogenation is 1 μm to 20 μm.
